(19) United States
(12) Patent Application Publication
(10) Pub. No.: US 2023/0333122 A1
(43) Pub. Date: Oct. 19, 2023

(54) INTERFERENCE MONITORING FOR PROVIDING A VERIFIED ANALYTE MEASUREMENT

(71) Applicant: Roche Diagnostics Operations, Inc., Indianapolis, IN (US)

(72) Inventors: Daniel Intelmann, München (DE); Peter Heiss, Penzberg (DE)

(21) Appl. No.: 18/333,347

(22) Filed: Jun. 12, 2023

Related U.S. Application Data
(63) Continuation of application No. PCT/EP2021/085314, filed on Dec. 10, 2021.

(30) Foreign Application Priority Data
Dec. 11, 2020 (EP) 20213481.3

(51) Int. Cl. G01N 33/96; G01N 30/88; G01N 33/74; G01N 30/72; G01N 30/86

(52) U.S. Cl. CPC G01N 33/743; G01N 30/72; G01N 30/8624; G01N 30/88; G01N 33/96; G01N 2030/8813; G01N 2333/575; G01N 2496/00

(57) ABSTRACT

The present invention relates to a method for providing a verified analyte measurement of a sample with a chromatography mass spectrometer device, said method comprising the following steps: a) admixing an interferent monitoring compound and, optionally an internal standard, to the sample; b) determining a chromatogram of the sample by acquiring a plurality of data points for signal intensities over time for said interferent monitoring compound, said analyte, and optionally said internal standard; and c) comparing a property of an interferent monitoring compound peak to a property of an internal standard peak and/or to a property of an analyte peak; and to methods and systems related thereto.

The present invention relates to a method for providing a verified analyte measurement of a sample with a chromatography mass spectrometer device, said method comprising the following steps: a) admixing an interferent monitoring compound and, optionally an internal standard, to the sample, b) determining a chromatogram of the sample by acquiring a plurality of data points for signal intensities over time for said interferent monitoring compound, said analyte, and optionally said internal standard; and c) comparing a property of an interferent monitoring compound peak to a property of an internal standard peak and/or to a property of an analyte peak; and to methods and systems related thereto.

### BACKGROUND ART

For chromatography-MS assays, ratios of peak areas are the common way for obtaining calculations or verifications. They are part of several international guidelines for validation of mass spectrometric assays, such as those by the CLSI (Clinical and Laboratory Standards Institute), the EMA (European Medicines Agency), or the GTFCh (German society for toxicological and forensic chemistry). For the quality assurance of an assay, non-extracted system suitability tests with spiked compounds, measured before the analytical run, have to fulfill acceptance requirements, such as minimal absolute peak areas or maximal retention time deviation from a target value. Within the analytical test series, quality control (QC) samples are then tested with a certain frequency and the calculated result checked versus an acceptance range. Moreover, retention time, peak width (given by the retention time difference between the peak boundaries), absolute peak area of the internal standard (ISTD), and the quantifier/qualifier peak area ratio of the analyte are usually monitored in each sample and should fulfill acceptance requirements of maximal deviations or certain cut-offs values.

Nonetheless, certain interferents, which may or may not be present in a sample, are notoriously difficult to separate from an analyte of interest in chromatography-MS; these are in particular structurally similar compounds such as diastereomers or enantiomers, which may arise e.g. as degradation products of the analyte of interest.

Moreover, the quality of an analytic run may be influenced by factors which are difficult to control, such as column aging effects, matrix effects, sample-specific interferences, and the like, such that it is usually not possible to ascertain whether a specific analytic run was interference-free, but rather this has to be extrapolated from the quality assurance measures described above.

### Problem to Be Solved

In accordance, improved methods for quality control of routine analytical LC-MS measurements are highly desirable.

### SUMMARY

The above problem is addressed by the methods, system, computer program product, computer or computer network, computer loadable data structure, computer program, and storage medium with the features of the independent claims. Advantageous embodiments which might be realized in an isolated fashion or in any arbitrary combinations are listed in the dependent claims.

### DETAILED DESCRIPTION

In accordance, the present invention relates to a method for providing a verified analyte measurement of a sample with a chromatography mass spectrometer device, said method comprising the following steps.
a) admixing an interferent monitoring compound and, optionally an internal standard, to the sample;
b) determining a chromatogram of the sample by acquiring a plurality of data points for signal intensities over time for said interferent monitoring compound, said analyte, and optionally said internal standard; and
c) comparing a property of an interferent monitoring compound peak to a property of an internal standard peak and/or to a property of an analyte peak.

In general, terms used herein are to be given their ordinary and customary meaning to a person of ordinary skill in the art and, unless indicated otherwise, are not to be limited to a special or customized meaning As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B. no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one” In the methods of the present invention, the indicated method steps may be performed in any order deemed appropriate by the skilled person, in an embodiment, however, are performed in the indicated order.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “about” relates to the indicated value with the commonly accepted technical precision in the relevant field, preferably relates to the indicated value ± 20%, more preferably ± 10%, most preferably ± 5% Further, the term “essentially” indicates that deviations having influence on the indicated result or use are absent, i.e. potential deviations do not cause the indicated result to deviate by more than ± 20%, more preferably ± 10%, most preferably ± 5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially air” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s). As referred to herein, measured and calculated parameters are described on an exemplary basis, as the skilled person understands, the parameters may be modified by standard mathematical operations, in particular by multiplication, division, addition, subtraction, reciprocal forming, scaling, and other operations known to the skilled person; in an embodiment, references are adjusted accordingly, in particular by applying the same mathematical operations. Measured and calculated parameters may also be used in the calculation of a score, which may be calculated on the basis of one or more parameter values, which may optionally be weighted, and/or by further mathematical operations in particular as specified above, e.g. scaling.

The method for providing a verified analyte measurement is an in vitro method. Moreover, it may comprise steps in addition to those explicitly mentioned above. For example, further steps may relate, e.g., to providing a sample for step a), or further calculations in steps b) and/ or c). Also, in an embodiment, the method comprises a further step b1) of performing peak identification of at least one interferent monitoring compound peak and of at least one analyte peak; in an embodiment said step b1) further comprises performing peak identification of at least one internal standard peak. In a further embodiment, the method further comprises step b2) determining at least one property of an interferent monitoring compound peak, at least one property of an internal standard peak and/or at least one property of an analyte peak. Moreover, one or more of the method steps may be assisted or performed by automated equipment. In an embodiment, in particular steps b) and c), and optionally b1) and b2), are performed by a processor, in particular by a computer, which may be configured as an evaluation device, as specified elsewhere herein. In an embodiment, the method for providing a verified analyte measurement further comprises in step a) acquiring a plurality of data points for an interferent monitoring compound and for an analyte, and optionally, for an internal standard. In an embodiment, the method comprises further step d) providing a verified analyte measurement based on comparison step c). Thus, the method for providing a verified analyte measurement typically is a quality control method which is, in an embodiment, part of a routine method of analyte measurement in a sample, in an embodiment a method of routine analyte measurement and/or of interference checking and/or interference monitoring.

As used herein, the term “analyte” relates to any chemical compound or group or compounds which shall be determined in a sample. In an embodiment, the analyte is a macromolecule, i.e. a compound with a molecular mass of more than 1000 u (i.e. more than 1 kDa). In a further embodiment, the analyte is a biological macromolecule, in particular a polypeptide, a polynucleotide, a polysaccharide, or a fragment of any of the aforesaid. In an embodiment, the analyte is a small molecule chemical compound, i.e. a compound with a molecular mass of at most 1000 u (1 kDa) In a further embodiment, the analyte is a chemical compound metabolized by a body of a subject, in particular of a human subject, or is a compound administered to a subject in order to induce a change in the subject’s metabolism. In a further embodiment, the analyte is a metabolite of a subject. Thus, in an embodiment, the analyte is a drug of abuse or a metabolite thereof, eg. amphetamine; cocaine; methadone; ethyl glucuronide; ethyl sulfate; an opiate, in particular buprenorphine, 6-monoacatylmorphine, codeine, dihydrocodeine, morphine, morphine-3-glucuronide, and/or tramadol, and/or an opioid, in particular acetylfentanyl, carfentanil, fentanyl, hydrocodone, norfentanyl, oxycodone, and/or oxymorphone. In an embodiment, the analyte is a therapeutic drug, e.g. valproic acid; clonazepam; methotrexate; voriconazole, mycophenolic acid (total); mycophenolic acid-glucuronide; acetaminophen; salicylic acid; theophylline, digoxin; an immuno suppressant drug, in particular cyclosporine, everolimus, sirclimus, and/or tacrolimus; an analgesic, in particular meperidine, normeperidine, tramadol, and/or O-desmethyl-tramadol; an antibiotic, in particular gentamycin, tobramycin, amikacin, vancomycin, piperacilline (tazobactam), meropenem, and/or linezolid; an antieplileptic, in particular phenytoin, valporic acid, free phenytoin, free valproic acid, levetiracetam, carbamazepine, carbamazepine-10,11-epoxide, phenobarbital, primidone, gabapentin, zonisamid, lamotrigine, and/or topiramate. In an embodiment, the analyte is a hormone. in particular cortisol, estradiol, progesterone, testosterone, 17-hydroxyprogesterone, aldosterone, dehydroepiandrosteron (DHEA), dehydroepiandrosterone sulfate (DHEA-S), dihydrotestosterone, and/or cortisone; in an embodiment, the sample is a serum or plasma sample and the analyte is cortisol, DHEA-S, estradiol, progesterone, testosterone, 17-hydroxyprogesterone, aldosterone, DHEA, dihydrotestosterone, and/or cortisone; in an embodiment, the sample is a saliva sample and the analyte is cortisol, estradiol, progesterone, testosterone, 17-hydroxyprogesterone, androstendione, and/or cortisone; in an embodiment, the sample is a urine sample and the analyte is cortisol, aldosterone, and/or cortisone. In an embodiment, the analyte is a vitamin, in an embodiment vitamin D. in particular ergocalciferol (Vitamin D2) and/or cholecalciferol (Vitamin D3) or a derivative thereof, e.g. 25-hydroxy-vitamine-D2, 25-hydroxy-vitamine-D3, 24,25-dihydroxy-vitamine-D2, 24,25-dihydroxy-vitamine-D3, 1.25-dihydroxy-vitamine-D2, and/or 1,25-dihydroxy-vitamine-D3. In an embodiment, the analyte is Vitamin D or is Testosterone.

The term “providing an analyte measurement” is understood by the skilled person to relate to any and all measurements providing data relating to determining the amount of an analyte in a sample, thus, providing an analyte measurement, in an embodiment, comprises providing data relating to analyte determination and/or providing data relating to measurement verification as specified herein below. As used herein, the term providing an analyte measurement relates to a determination of the analyte based on chromatography of the analyte on the chromatography unit of the chromatography mass spectrometer device as specified elsewhere herein. In an embodiment, the term includes a qualitative, semi-quantitative, or quantitative determination of the amount of analyte in a sample, in an embodiment relates to a quantitative determination of the amount of analyte in a sample. Methods for determining the amount of an analyte by chromatography, in an embodiment by chromatography-MS are, in principle, known to the skilled person. In an embodiment, the method comprises quantitatively determining an amount of an analyte in a sample by determining a peak, area of the analyte (analyte peak area). In an embodiment, further a peak area of an internal standard is determined (IS peak area) and a ratio of the analyte peak area and the IS peak area is determined and, in an embodiment, said ratio is compared to a calibration function, whereby a concentration value is determined. Corresponding methods are known to the skilled person. In an embodiment, providing an analyte measurement may, however, also comprise, in an embodiment consist of, determining data for verifying said analyte measurement as specified herein below, and, in accordance, may lack qualitative, semi-quantitative, or quantitative determination of the amount of analyte in a sample: this may be the case e.g. if it is found that the measurement is rejected in the verification as specified herein below.

The term “providing a verified analyte measurement”, as used herein, relates to providing an analyte measurement for which the risk of interference by a non-analyte compound is assessed or can be assessed, in an embodiment quantified. Thus, providing a verified analyte measurement may be determining that an analyte measurement is rejected, i.e. determining that the risk that the analyte measurement was confounded by an interferent is inacceptably high; or may be determining that an analyte measurement is accepted, i.e. determining that the risk that the analyte measurement was confounded by an interferent is acceptable or that there is no such risk. In an embodiment, providing a verified analyte measurement comprises excluding interference by an interferent, in an embodiment excluding interference by an interferent as specified herein below, i.e., in an embodiment a verified analyte measurement is a measurement for which it is assured that the analyte measurement was not confounded by the interferent, i.e. is an accepted analyte measurement. In accordance, the verification of the analyte measurement in an embodiment pertains to evaluating whether the analysis would have separated the analyte from potential interferents, if present, at least to a pre-defined acceptable degree, in an embodiment to an extent making a reliable measurement of the analyte possible. In accordance with the above, the method for providing a verified analyte measurement may be preceded, may comprise, and/or may be followed by a further step of providing an analyte measurement. It will, however, be understood that in case an analyte measurement is rejected, providing a verified analyte measurement may comprise only reporting rejection of the measurement, but may e.g. lack determining and/or reporting the amount of the analyte. In an embodiment, the analyte measurement is accepted in case the resolution between the analyte peak and the interferent monitoring compound peak or between the internal standard peak and the interferent monitoring compound peak calculated according to formula (1) described herein below is higher than 1.5, in an embodiment higher than 2 Possible other criteria for such sufficient separation are also provided elsewhere herein. In an embodiment, in case analyte measurement is not accepted, the analyte measurement value is not reported, the analyte measurement is flagged as not rejected, and/or the chromatography device is flagged unsuitable for measurement of the analyte in the sample type.

The term “chromatography mass spectrometer device”, abbreviated as “chromatography-MS device”, is understood by the skilled person. In an embodiment, the term relates to a device configured for performing a combination of chromatography with mass spectrometry (MS). Thus, the device, in an embodiment, comprises at least one chromatography unit, and at least one MS unit, wherein the chromatography unit and the MS unit are coupled via at least one interface, The chromatography unit, in an embodiment, is a liquid chromatography (LC) unit, a gas chromatography (GC) unit, a capillary electrophoresis chromatography unit, or an ion mobility chromatography unit, in a further embodiment, is an LC unit. The aforesaid chromatography types are known to the skilled person, as are principal chromatography-MS methods and devices. As used herein, the term “liquid chromatography (LC) unit”, in an embodiment, relates to an analytical module configured to separate one or more analytes of interest of a sample from other components of the sample via liquid chromatography, in an embodiment for detection of the one or more analytes with the mass spectrometry device. The LC may be based on any separation principle deemed appropriate by the skilled person; in an embodiment, the LC is reverse phase chromatography, hydrophobic interaction chromatography, ion exchange chromatography, size exclusion chromatography, affinity chromatography, or chiral chromatography; in a further embodiment, the LC is reverse phase chromatography. The LC device may comprise at least one LC column. For example, the LC device may be a single-column LC device or a multi-column LC device having a plurality of LC columns. The LC column may have a stationary phase through which a mobile phase is pumped in order to separate and/or elute and/or transfer the analyte(s) of interest. The LC unit may be or may comprise at least one high-performance liquid chromatography (HPLC) unit and/or at least one micro liquid chromatography (µLC) device As used herein, the term “mass spectrometry unit”, in an embodiment, relates to a mass analyzer configured for detecting at least one analyte based on a mass to charge ratio (m/z) of the analyte, the interferent monitoring compound, or the internal standard, or a fragment thereof. The mass spectrometry unit may be or may comprise at least one quadrupole mass spectrometry device. The interface coupling the LC unit and the MS unit may comprise at least one ionization source configured for generating molecular ions and for transferring the molecular ions into the gas phase. In an embodiment, the MS unit is a tandem mass spectrometry (MS/MS) unit, in a further embodiment, a triple quadrupole MS/MS, in a further embodiment in Multiple Reaction Monitoring (MRM) mode.

According to step a) of the method, an interferent monitoring compound and, optionally an internal standard, is admixed to the sample to be analyzed. The term “admixing” is understood by the skilled person to relate to adding to a sample an interferent monitoring compound and optionally an internal standard in a manner making its or their detection by the chromatography-MS protocol used possible. Admixing may be before, during, and/or after, any sample preparation steps. The interferent monitoring compound and the optional internal standard may be added simultaneously or concomitantly, in an embodiment are admixed simultaneously. It is, however also envisaged that the interferent monitoring compound may be admixed shortly before chromatography, while the internal standard may be admixed e.g. before any sample preparation steps. Thus, the sample may be spiked with the internal standard. The interferent monitoring compound and the optional internal standard may be added to the sample at predefined concentrations. The concentrations of the interferent monitoring compound and the optional internal standard may be non-identical, may be pre-determined and significantly higher than the assumed concentrations of the interferent and the analyte, respectively.

The term “interferent” is used herein in a broad sense to relate to any compound potentially present in a sample and potentially interfering with correct determination of an analyte on a chromatography-MS device. Thus, the interferent in an embodiment is a compound known or suspected to be potentially present in a sample, thus, for the avoidance of doubt, the interferent does not have to be present in a specific sample. Thus, in an embodiment, the method for providing a verified analyte measurement provides an evaluation whether the interferent would have been separated from the analyte during analysis if present and does not necessarily provide information on whether an interferent was actually present in a sample. Thus, in an embodiment, the interferent is selected to be the compound or one of the compounds most difficult to separate from the analyte in a given analysis protocol. In accordance, verifying that the interferent would have been separated from the interferent, if present, in an embodiment is deemed indicative that the analyte measurement was not confounded by any interfering compound; i.e., in an embodiment, the interferent is used as a surrogate marker of interference. The interferent, in an embodiment has similar physico-chemical properties as the analyte, in a further embodiment has similar or identical elution properties as the analyte in chromatography and/or has a similar or identical fragmentation pattern in MS. In an embodiment, the interferent is a compound having a retention time in the chromatography used in the analysis protocol corresponding to the retention time of the analyte ±20%, in an embodiment ±10%. In an embodiment, the interferent is an isobaric compound of the analyte. In an embodiment, the resolution between the analyte peak and the interferent peak in the chromatography used in the analysis protocol is less than 3, in an embodiment less than 2, in a further embodiment is of from 1 to 3, in an embodiment of from 1.5 to 2.5. In an embodiment, the interferent is a compound structurally similar to the analyte, in a further embodiment is an isomer of the analyte, in a further embodiment a stereoisomer of the analyte, in a further embodiment a diastereomer, an enantiomer, or a cis-trans isomer. In an embodiment, the diastereomer is an epimer or an anomer, in an embodiment an epimer. Thus, in an embodiment, the analyte is Vitamin D and, in an embodiment, the interferent is Epi-Vitamin D, also in an embodiment, the analyte is Testosterone and, in an embodiment, the interferent is Epitestosterone.

As used herein, the term “interferent monitoring compound” relates to a compound having similar, in an embodiment identical, physico-chemical properties as the interferent. Thus, in an embodiment the interferent monitoring compound has a similar chemical structure, in a further embodiment an identical chemical structure, compared to the interferent. In an embodiment, the interferent monitoring compound is the interferent or an isotope-labelled derivative thereof, in a further embodiment is an isotopologue of the interferent. Thus, in case the interferent is Epi-Vitamin D, the interferent monitoring compound may be ¹³C₅-2S-Hydroxy-Vitamin D3 epimer, ¹³C.Epi-Vitamin D, or ²H₃-Epi-Vitamin D (“D3-Epi-Vitamin D”) Also in an embodiment, in case the interferent is Epitestosterone, the interferent monitoring compound may be Epitestouterone-¹³C₃ or Epitestosterone-D₃.

The term “internal standard” is understood by the skilled person. In an embodiment, the internal standard has the analogous physico-chemical and/or structural relationship to the analyte as is specified herein above for the relationship of the interferent monitoring compound to the interferent.

As used herein, the term “sample”, also referred to as “test sample”, relates to any type of composition of matter; thus, the term may refer, without limitation, to any arbitrary sample such as a biological sample. In an embodiment, the sample is a liquid sample, in a further embodiment an aqueous sample. In an embodiment, the test sample is selected from the group consisting of: a physiological fluid, including blood, serum, plasma, saliva, ocular lens fluid, lacrimal fluid, cerebrospinal fluid, sweat, urine, milk, ascites, mucus, synovial fluid, peritoneal fluid, and amniotic fluid; lavage fluid; tissue, cells, and the like. In an embodiment, the sample is a blood, plasma, serum, saliva, or urine sample, in a further embodiment a blood, plasma, or serum sample, in a further embodiment a serum or plasma sample. The sample may, however, also be a natural or industrial liquid, in particular surface or ground water, sewage, industrial wastewater, processing fluid, soil eluates, and the like. In an embodiment, the sample comprises or is suspected to comprise at least one chemical compound of interest, i.e. a chemical which shall be determined, which is referred to as “analyte”. The sample may comprise or be suspected to comprise one or more interferents as specified herein above. The sample may comprise one or more further chemical compounds, which are not to be determined and which are commonly referred to as “matrix”. The sample may be used directly as obtained from the respective source or may be subjected to one or more pretreatment and/or a sample preparation step(s). Thus, the sample may be pretreated by physical and/or chemical methods, in an embodiment by centrifugation, filtration, mixing, homogenization, chromatography, precipitation, dilution, concentration, contacting with a binding and/or detection reagent, and/or any other method deemed appropriate by the skilled person.

According to step b) of the method, a chromatogram of the sample is determined by acquiring a plurality of data points for signal intensities over time for said interferent monitoring compound, said analyte, and optionally said internal standard.

The term “chromatogram” is known to the skilled person. In an embodiment, the term relates to a correlation plot of a quantitative measure of one or more signals obtained from a sample by an MS detector with the progress of a chromatographic separation, in an embodiment over time, e.g. retention time and/or elution volume. In an embodiment, said quantitative measure of signal(s) correlates with the concentration of at least part of sample constituents, in particular with the analyte, the internal standard and/ or the interferent monitoring compound; thus, the quantitative measure of signals may in particular be a signal intensity Thus, in an embodiment, the chromatogram is an MS chromatogram, in a further embodiment an MS/MS chromatogram. The signal measured, in an embodiment, is an abundance of an ion (in an embodiment measured as intensity at its m/z ratio), a fragmentation pattern (e.g. measured as intensity of at least two fragments generated), or as at least one multiple reaction monitoring transition (e.g. measured as the intensity of at least one daughter ion generated from a predetermined parent ion). In an embodiment, said quantitative measure of signals comprises an analyte signal intensity, an interferent monitoring compound intensity and/or an internal standard signal intensity. In an embodiment, the quantitative measure of signals comprises an analyte quantifier, an interferent monitoring compound quantifier, an internal standard quantifier, an analyte qualifier, an interferent monitoring compound qualifier, and/or an internal standard qualifier. Thus, in an embodiment, determining at least one chromatogram comprises measuring an analyte quantifier and an interferent monitoring compound quantifier, and optionally an internal standard quantifier and/or an internal standard qualifier; or determining at least one chromatogram comprises measuring an analyte quantifier and an interferent monitoring compound qualifier, and optionally an internal standard quantifier and/or an internal standard qualifier; or determining at least one chromatogram comprises measuring an analyte qualifier and an interferent monitoring compound quantifier, and optionally an internal standard quantifier and/or an internal standard qualifier, or determining at least one chromatogram comprises measuring an analyte qualifier and an interferent monitoring compound qualifier, and optionally an internal standard quantifier and/or an internal standard qualifier; in such cases, in an embodiment, the MS is tandem MS. As will be understood by the skilled person, the aforesaid representation may be, but does not have to be, a graphical representation; the representation may, however, also be provided e.g. as a list of value pairs, e.g. elution ti me/quantifier value pairs and/or elution time/qualifier value pairs, or as a mathematical model. As indicated above, the chromatogram may represent more than one signal, in an embodiment, the chromatogram represents two signals; in a further embodiment, the chromatogram represents three signals, in an embodiment as specified herein below. As is understood by the skilled person in particular in view of the description herein, more than one signal may be determined for each of the analyte, the interferent monitoring compound, and/or the internal standard; e.g. a quantifier and a qualifier may be determined for each of the aforesaid compounds. Thus, in a further embodiment, the chromatogram represents more than three signals, e.g. four, five, six, or even more than six signals.

As will be understood, the chromatogram may as well represent further signals; the aforesaid multitude of signals may, however, also be represented by a multitude of chromatograms representing one signal each. As the skilled person further understands, elution time may be replaced by any other measure of chromatography progress deemed appropriate by the skilled person, in particular by elution volume or by retention time. The chromatogram may comprise data points over the whole chromatography-MS run of a sample; in an embodiment, in particular in case the location of the analyte peak and/or the interferent monitoring compound peak in the chromatogram can be predicted, e g. from previous runs, the chromatogram may comprise data points over the expected analyte peak breadth and the expected interferent monitoring compound peak, e.g. from the putative lower peak boundary of the analyte peak to the putative upper peak boundary of the interferent monitoring compound peak, or vice versa, optionally further including data extending 1% in an embodiment 5%, in a further embodiment 10%, in a further embodiment 50%, in a further embodiment 100% of the respective putative boundary value downstream and/or upstream of the respective peak.

In an embodiment, the chromatogram is determined based on signals which are non-identical between the analyte, the internal standard, and the interferent monitoring compound, i.e. in an embodiment are different for all three compounds. Thus, in an embodiment, the signal(s) determined for the analyte is/are different from the signal(s) determined for the interferent monitoring compound and both the signal(s) determined for the analyte and the signal(s) determined for the interferent monitoring compound are different from the signal(s) determined for the internal standard. Thus, in an embodiment, a chromatogram comprises at least three correlation plots of quantitative measures of signals, at least one each for the analyte, for the interferent monitoring compound, and for the internal standard, determined by an MS detector with the progress of a chromatographic separation. In an embodiment, the internal standard and the interferent monitoring compound are isotopically labeled in such case.

In an embodiment, the chromatogram is determined in step b) based on signals which are non-identical between the analyte and the internal standard; optionally, in such case, the signals are identical between the internal standard and the interferent monitoring compound. Thus, in an embodiment, the signal(s) determined for the internal standard optionally is/are identical to the signal(s) determined for the interferent monitoring compound and both the signal(s) determined for the analyte and the signal(s) determined for the interferent monitoring compound are different from the signal(s) determined for the internal standard. Thus, in an embodiment, a chromatogram comprises two correlation plots of quantitative measures of signals, one for the internal standard and the interferent monitoring compound, and one for the analyte, determined by an MS detector with the progress of a chromatographic separation. In an embodiment, the internal standard and the interferent monitoring compound are isotopically labeled in such case.

In an embodiment, the chromatogram is determined in step b) based on signals which are non-identical between the analyte and the interferent monitoring compound. Thus, in an embodiment, the signal(s) determined for the interferent monitoring compound is/are different from the signal(s) determined for the analyte, and (a) signal(s) for the internal standard is/are optionally not determined. Thus, in an embodiment, a chromatogram comprises two correlation plots of quantitative measures of signals, one for the interferent monitoring compound, and one for the analyte, determined by an MS detector with the progress of a chromatographic separation. In an embodiment, the interferent monitoring compound is isotopically labeled in such case.

In an embodiment, the chromatogram is determined in step b) based on signals which are non-identical between the internal standard and the interferent monitoring compound. Thus, in an embodiment, the signal(s) determined for the interferent monitoring compound is/are different from the signal(s) determined for the internal standard, and (a) signal(s) for the analyte may optionally not be determined, but is determined in an embodiment Thus, in an embodiment, a chromatogram comprises two correlation plots of quantitative measures of signals, one for the interferent monitoring compound and optionally the analyte, and one for the internal standard, determined by an MS detector with the progress of a chromatographic separation. In an embodiment, the internal standard is isotopically labeled in such case.

In an embodiment, the chromatogram is determined in step b) based on signals which are identical between the analyte and the interferent monitoring compound. Thus, in an embodiment, the signal(s) determined for the analyte is/are identical to the signal(s) determined for the interferent monitoring compound. Thus, in an embodiment, a chromatogram comprises a correlation plot of quantitative measures of signals for the analyte and the interferent monitoring compound. In an embodiment, the interferent monitoring compound is not isotopically labeled in such case.

According to step c) of the method, a property of an interferent monitoring compound peak is compared to a property of an internal standard peak and/or to a property of an analyte peak.

The term “peak” is known to the skilled person and, in an embodiment, relates to at least one local maximum of a chromatogram. In accordance, the term “analyte peak” relates to a peak correlating with an analyte, in an embodiment for an identified peak of the analyte of interest; the term “interferent monitoring compound peak” relates to a peak correlating with an interferent monitoring compound, in an embodiment for an identified peak of the interferent monitoring compound; and the term “internal standard peak” relates to a peak correlating with an internal standard, in an embodiment for an identified peak of the internal standard. Methods for peak detection and peak integration are known in the art, the term “peak integration”, in an embodiment, relating to at least one mathematical operation and/or mathematical algorithm for determining a peak area enclosed by a peak of the chromatogram. Specifically, the integration of the peak may comprise identification and/or measurement of curve characteristics of the chromatogram. The peak integration may comprise one or more of peak detection, peak finding, peak identification, peak fitting, peak evaluation, determining a lower peak boundary and/or an upper peak boundary, determining of background, and determining of basis line. The peak integration may allow determining of one or more of peak area, retention time, peak height, and peak width. In an embodiment, peak detection and/or peak integration are performed automatically, i.e. without manual action or interaction with a user. In particular, peak identification and/or peak detection and/or determining of peak area may be performed non-manually and without manual action or interaction with a user. The term “peak identification”, as used herein, relates to any measure determining at least one parameter of a peak in a chromatogram. In an embodiment, said identification comprises identifying a lower peak boundary and/or an upper peak boundary, identifying a peak maximum, identifying peak identity and/or peak purity, and/or identifying an analyte peak area to internal standard peak area ratio (peak area ratio). In accordance with the above, in an embodiment, the internal standard peak is an internal standard quantifier peak or an internal standard qualifier peak, said interferent monitoring compound peak is an interferent monitoring compound quantifier peak or an interferent monitoring compound qualifier peak, and/or said analyte peak is an analyte quantifier peak or an analyte qualifier peak.

The term “peak property” includes any and all ascertainable properties of a peak of a chromatogram, in particular those indicated herein above, e.g. a lower peak boundary, an upper peak boundary, a peak maximum, a peak height, a peak width at baseline, a full peak width at half maximum, and the like. In an embodiment, for comparing a property of an interferent monitoring compound peak to a property of an internal standard peak and/or to a property of an analyte peak in step c), in particular a peak property is selected to allow establishing whether an analyte peak and/or an internal standard peak was sufficiently separated from an interferent monitoring compound peak. Accordingly, e.g. in case the analyte elutes before the interferent monitoring compound, the upper peak boundary of the analyte peak may be compared to the lower peak boundary of the interferent monitoring compound peak; for this purpose, e.g. elution times may be compared, and the analyte measurement may e.g. be accepted in case the difference between the elution time of the upper analyte peak boundary and the lower interferent monitoring compound peak boundary is above a pre-defined value. e.g. is at least 0. For the case that the analyte elutes after the interferent monitoring compound, the above applies mutatis mutandis. Thus, peak properties may be directly compared in step c) as deemed appropriate by the skilled person. In an embodiment, the comparing in step c) may further comprise providing derivative values based on the aforesaid peak properties. In particular, a difference in retention time may be calculated as a difference between the elution time of the maximum of a first peak, e.g. the analyte peak and/or the internal standard peak, and the maximum of a second peak, e.g. an interferent monitoring compound peak. In an embodiment, resolution between the analyte peak and the interferent monitoring compound peak and/or resolution between the internal standard peak and the interferent monitoring compound peak is calculated, in an embodiment according to formula (1):

$$R = 2 \frac{{t}_{2} - {t}_{1}}{{w}_{2} + {w}_{1}}$$

with R = resolution,
t₁ = retention time of the first peak,
t₂= retention time of the second peak,
w₁= full width at half maximum of the first peak; and
w₂ = full width at half maximum of the second peak.

As is understood by the skilled person, in step c), in an embodiment, corresponding peak properties are compared, i.e. a retention time may be compared to a retention time, an elution volume of an upper peak boundary may be compared to an elution time of a lower peak boundary, and the like, or a parameter derived from one or more of such peak property or properties may be calculated. Also, peak properties are compared between peaks so as to allow a verified analyte measurement to be provided. Thus, in an embodiment, in step c) a property of an internal standard peak is compared to a property of an interferent monitoring compound peak; and/or in step c) a property of an analyte peak is compared to a property of an interferent monitoring compound peak. The comparison of step c) may comprise comparison of the respective peaks, in particular with regards to a potential overlap between the peaks. In an embodiment, the comparison of step c) comprises comparing retention times of the respective peaks, optionally additionally taking into account peak width; in a further embodiment, the comparison of step c) comprises calculating a resolution, in an embodiment as specified herein above an comparing said resolution to at least one acceptance criterion.

As used herein, the term “amount” of an analyte relates to any quantitative measure of the analyte, and is equivalent to other corresponding measures such as mass fraction and concentration, which can be calculated from the amount in case sample mass or sample volume is known. Thus, the result of measurement of an analyte in a sample may be expressed in any unit deemed appropriate by the skilled person, including arbitrary units, measures of weight, of mass fraction, of concentration, and the like, or measures derived therefrom, e.g. international units according to a pre-defined definition.

The term “subject”, as used herein, relates to an animal, in an embodiment a vertebrate, in a further embodiment a mammal, in a further embodiment a human. In an embodiment, the subject is known or suspected to comprise the analyte as specified elsewhere herein. In an embodiment, the subject is a patient, i.e. a subject under medical examination and/or treatment. Advantageously, it was found in the work underlying the present invention that by including an interferent monitoring compound as specified into routine analyte measurements, it can be established on the fly whether a particular measurement is acceptable, thus enabling direct quality control for each sample measurement and reducing the effort for additional quality control measures.

The definitions made above apply mutatis mutandis to the following Additional definitions and explanations made further below also apply for all embodiments described in this specification mutatis mutandis.

The present invention further relates to a method of quality control of a chromatography mass spectrometry measurement of an analyte in a sample, comprising the steps
A) measuring the analyte in the sample using the chromatography mass spectrometer device and determining at least one chromatogram;
B) verifying the analyte measurement according to the method for providing a verified analyte measurement of a sample as specified herein above, and
C) evaluating quality of said chromatography mass spectrometry measurement based on the results of step B).

The method of quality control is an in vitro method. Moreover, it may comprise steps in addition to those explicitly mentioned above. For example, further steps may relate, e.g., to providing a sample for step A), or further calculations in steps B) and/ or C). Also, the method may comprise a further step in case the analyte measurement is rejected in step B), in particular as specified elsewhere herein. The method may be assisted or performed by automated equipment, e.g. an evaluation device as specified herein below. In particular, method steps B) and/or C), in an embodiment steps B) and C), may be performed by a computer.

The term “quality control”, as used herein, is known to the skilled person. In an embodiment, quality control is the process of ensuring that processes performed and/or goods or measurements produced by an entity are in conformity with pre-defined quality criteria. In a further embodiment, quality control in sample measurement, in particular in measurement of medical samples such as patient samples, e.g. in clinical diagnostics and/or clinical chemistry, comprises ensuring that the analysis results obtained with a specific measuring method correspond to the results obtainable with a gold standard method and, therefore, in an embodiment correspond to the results theoretically obtainable, within a pre-specified range. Thus, in an embodiment, the method for quality control comprises as further step evaluating quality of said measurement based on the results of step B), and optionally taking appropriate measures, in an embodiment as specified herein above. In an embodiment, in case the analyte measurement is accepted, it is output to a user; in case the analyte measurement is rejected, at least one of (i) flagging the result of step A) as unreliable; (ii) not outputting the result of step A), and (iii) flagging the chromatography device as unsuitable for measurement of the analyte in the sample type is performed.

The term “measuring an analyte in a sample” is understood by the skilled person, in particular in view of the explanations herein above. As the skilled person understands, measuring an analyte in a sample may be part of method step B). The result of measurement of an analyte in a sample may be expressed in any unit deemed appropriate by the skilled person, including arbitrary units, measures of weight, of mass fraction, of concentration, and the like, or measures derived therefrom, e.g. international units according to a pre-defined definition.

The present invention also relates to a system for determining an amount of at least one analyte in a sample comprising:
(I) at least one chromatography mass spectrometer device, wherein the chromatography mass spectrometer device is configured for measuring the analyte in the sample and for acquiring data points over time, in an embodiment for performing step b) of a method as specified herein above; and
(II) at least one evaluation device, wherein the evaluation device is configured for performing at least step c) of a method as specified herein above.

The term “system”, as used herein, relates to different means which are operatively linked to each other. Said means may be implemented in a single physical unit or may be physically separated units which are operatively linked to each other. Suitable components and their properties are described elsewhere herein below and also herein above in the context of the methods described. Consequently, the methods of the present invention can be implemented by the system specified herein. Thus, in an embodiment, the device is configured to perform the method for providing a verified analyte measurement as specified elsewhere herein, and/or the method of quality control as specified elsewhere herein. The system may comprise further devices or units, in particular a data collector, an output unit, a communication interface, and/or any other device or unit deemed appropriate by the skilled person.

The chromatography mass spectrometer device and means and methods for determining at least one chromatogram have been described herein above in the context of the methods of the present invention.

The term “evaluation device” generally refers to an arbitrary device adapted to perform the method step(s) as described above, in an embodiment by using at least one data processing device and, in a further embodiment, by using at least one processor and/or at least one application-specific integrated circuit. Thus, as an example, the at least one evaluation device may comprise at least one data processing unit having a software code stored thereon comprising a number of computer commands. The evaluation device may provide one or more hardware elements for performing one or more of the indicated operations and/or may provide one or more processors with software running thereon for performing one or more of the method steps.

As used herein, the term “data collector” relates to any arbitrary storage unit configured for storing data, in particular data points determined by the chromatography mass spectrometer device, chromatograms, peak properties, results of peak identification and/or verifications, and/or recommendations provided and/or decisions taken on further proceeding with regards to the sample. In an embodiment, the data collector comprises at least one database configured for receiving and/or storing at least one chromatogram. In an embodiment, the data collector comprises at least one database comprising one or more reference values.

The term “output unit”, as used herein, relates to any arbitrary unit configured for a transfer of information from the system to another entity, wherein another entity may be a further data processing device and/or a user. Thus, the output device may comprise a user interface, such as an appropriately configured display, or may be a printer. The output unit may, however also be an indicator, e.g. an indicator lamp, indicating e.g. that the analyte measurement should be rejected, or a communication interface.

The term “communication interface” is understood by the skilled person to relate to any arbitrary interface configured for exchange of information, in particular exchange of data. Such data exchange may be achieved by a permanent or temporary physical connection, such as coaxial, fiber, fiber-optic or twisted-pair, 10 BASE-T cables, storage unit connectors, such as USB, firewire, and similar connectors. Alternatively, it may be achieved by a temporary or permanent wireless connection using. e.g., radio waves, such as Wi-Fi, LTE, LTE-advanced or Bluetooth.

The invention further discloses and proposes a computer program including computer-executable instructions for performing a method according to the present invention in one or more of the embodiments enclosed herein when the program is executed on a computer or computer network. Specifically, the computer program may be stored on a computer-readable data carrier. Thus, specifically, one, more than one or even all method steps as indicated above may be assisted or performed by using a computer or a computer network, preferably by using a computer program.

The invention further discloses and proposes a computer program product having program code means, in order to perform the method according to the present invention in one or more of the embodiments enclosed herein when the program is executed on a computer or computer network. Specifically, the program code means may be stored on a computer-readable data carrier.

Further, the invention discloses and proposes a data carrier having a data structure stored thereon, which, after loading imo a computer or computer network, such as into a working memory or main memory of the computer or computer network, may execute the method according to one or more of the embodiments disclosed herein.

The invention further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Further, the invention proposes and discloses a modulated data signal which contains instructions readable by a computer system or computer network, for performing the method according to one or more of the embodiments disclosed herein.

In an embodiment, referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, the present invention further discloses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network.
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

In view of the above, the following embodiments are particularly envisaged:

Embodiment 1: A method for providing a verified analyte measurement of a sample with a chromatography mass spectrometer device, said method comprising the following steps:
a) admixing an interferent monitoring compound and, optionally an internal standard, to the sample;
b) determining a chromatogram of the sample by acquiring a plurality of data points for signal intensities over time for said interferent monitoring compound, said analyte, and optionally said internal standard, and
c) comparing a property of an interferent monitoring compound peak to a propeny ofan internal standard peak and/or to a property of an analyte peak.

Embodiment 2: The method of embodiment 1, wherein said interferent monitoring compound and/or said internal standard is/are isotope-labelled.

Embodiment 3: The method of embodiment 1 or 2, wherein said internal standard is an isotopologue of the analyte and/or wherein said interferent monitoring compound is an isotopologue of an interferent

Embodiment 4: The method of any one of embodiments 1 to 3, wherein in step a) an interferent monitoring compound and an internal standard are admixed to the sample.

Embodiment 5: The method of any one of embodiments 1 to 3, wherein in step a) an interferent monitoring compound is admixed to the sample

Embodiment 6. The method of any one of embodiments 1 to 5, wherein in step b) the chromatogram is determined based on signals being (1) non-identical between the analyte, the internal standard, and the interferent monitoring compound; (II) identical between the internal standard and the interferent monitoring compound and non-identical between the analyte and the internal standard, (III) non-identical between the analyte and the interferent monitoring compound. (IV) non-identical between the internal standard and the interferent, or (V) identical between the analyte and the interferent monitoring compound.

Embodiment 7: The method ofany one ofembodiments 1 to 6, wherein in step c) (i) the property of the internal standard peak is compared to the property of the interferent monitoring compound peak, and/or (ii) the property of the analyte peak is compared to the property of the interferent monitoring compound peak

Embodiment 8. The method of any one of embodiments 1 to 7, wherein
A) in step a) an interferent monitoring compound and an internal standard are admixed to the sample; wherein said interferent monitoring compound and said internal standard are isotope-labelled, in an embodiment wherein said internal standard is an isotopologue of the analyte and/or wherein said interferent monitoring compound is an isotopologue of an interferent;
B) in step b) the chromatogram is determined based on signals being non-identical between the analyte, the internal standard, and the interferent monitoring compound; and C) wherein in step c) the property of the internal standard peak is compared to the property of the interferent monitoring compound peak.

Embodiment 9: The method of any one of embodiments 1 to 7, wherein
A) in step a) an interferent monitoring compound and an internal standard are admixed to the sample; wherein said interferent monitoring compound and said internal standard are isotope-labelled, in an embodiment wherein said internal standard is an isotopologue of the analyte and/or wherein said interferent monitoring compound is an isotopologue of an interferent;
B) in step b) the chromatogram is determined based on signals being identical between the internal standard and the interferent monitoring compound and non-identical between the analyte and the internal standard; and
C) wherein in step c) the property of the internal standard peak is compared to the property of the interferent monitoring compound peak.

Embodiment 10: The method of any one of embodiments 1 to 7, wherein
A) in step a) an interferent monitoring compound is admixed to the sample; wherein said interferent monitoring compound is isotope-labelled, in an embodiment wherein said interferent monitoring compound is an isotopologue of an interferent;
B) in step b) the chromatogram is determined based on signals being non-identical between the analyte and the interferent monitoring compound; and
C) wherein in step c) the property of the analyte peak is compared to the property of the interferent monitoring compound peak.

Embodiment 11: The method of any one of embodiments 1 to 7, wherein
A) in step a) an interferent monitoring compound and an internal standard are admixed to the sample: wherein said internal standard is isotope-labelled, in an embodiment wherein said internal standard is an isotopologue of the analyte;
B) in step b) the chromatogram is determined based on signals being non-identical between the internal standard and the interferent monitoring compound; and
C) wherein in step c) the property of the internal standard peak is compared to the property of the interferent monitoring compound peak.

Embodiment 12: The method of any one of embodiments 1 to 7, wherein
A) in step a) an interferent monitoring compound is admixed to the sample:
B) in step b) the chromatogram is determined based on signals being identical between the analyte and the interferent monitori ng compound; and
C) wherein in step c) the property of the analyte peak is compared to the property of the interferent monitoring compound peak.

Embodiment 13: The method of any one of embodiments 1 to 12, wherein said method further comprises performing peak identification of at least one interferent monitoring compound peak, at least one analyte peak and, optionally, at least one internal standard peak.

Embodiment 14: The method of embodiment 13, wherein said analyte measurement is based on the at least one analyte peak identified.

Embodiment 15: The method of any one of embodiments 1 to 14, wherein said comparing in step c) comprises determining the retention times of at least one analyte peak, of at least one interferent monitoring compound peak, and, optionally of at least one internal standard peak.

Embodiment 16: The method of any one of embodiments 1 to 15, wherein said comparing in step c) comprises determining the peak width values of at least one analyte peak, of at least one interferent monitoring compound peak, and, optionally of at least one internal standard peak.

Embodiment 17: The method of any one of embodiments 1 to 16, wherein said comparing in step c) comprises determining a resolution between the analyte peak and the interferent monitoring compound peak or between the internal standard peak and the interferent monitoring compound peak, wherein, in an embodiment, resolution is calculated based on the retention times and the full width at half maximum values of the respective peaks, in a further embodiment according to formula (1):

$$R = 2 \frac{{t}_{2} - {t}_{1}}{{w}_{2} + {w}_{1}}$$

with R = resolution,
t₁ = retention time of the first peak,
t₂ = retention time of the second peak,
w₁ = full width at half maximum of the first peak, and
w₂ = full width at half maximum of the second peak.

Embodiment 18. The method of any one of embodiments 1 to 17, wherein said method comprises additional step d) providing a verified analyte measurement based on comparison step c), wherein, in an embodiment the analyte measurement is accepted in case the resolution between the analyte peak and the interferent monitoring compound peak or between the internal standard peak and the interferent monitoring compound peak is higher than 1.5, in an embodiment higher than 2.

Embodiment 19: The method of any one of embodiments 1 to 18, wherein said internal standard peak is an internal standard quantifier peak or an internal standard qualifier peak, wherein said interferent monitoring compound peak is an interferent monitoring compound quantifier peak or an interferent monitoring compound qualifier peak, and/or wherein said analyte peak is an analyte quantifier peak or an analyte qualifier peak.

Embodiment 20: The method of any one of embodiments 1 to 19, wherein said interferent is a compound having a retention time in chromatography corresponding to the retention time of the analyte ±20%, in an embodiment ±10%.

Embodiment 21: The method of any one of embodiments 1 to 20, wherein the resolution between the analyte peak and the interferent peak in chromatography is less than 3. in an embodiment less than 2.

Embodiment 22: The method of any one of embodiments 1 to 21, wherein the resolution between the analyte peak and the interferent peak in chromatography is of from 1 to 3, in an embodiment of from 1.5 to 2.5.

Embodiment 23: The method of any one of embodiments 1 to 22, wherein said interferent is a compound structurally similar to the analyte.

Embodiment 24: The method of any one of embodiments 1 to 23, wherein said interferent is an isomer of the analyte, in an embodiment a stereoisomer of the analyte, in a further embodiment a diastereomer, an enantiomer, or a cis-trans isomer.

Embodiment 25: The method of any one of embodiments 1 to 24, wherein said diastereomer is an epimer or an anomer, in an embodiment an epimer.

Embodiment 26: The method of any one of embodiments 1 to 25, wherein said analyte is Vitamin D and, in an embodiment, the interferent is Epi-Vitamin D.

Embodiment 27: The method of any one of embodiments 1 to 26, wherein said interferent monitoring compound is an isotopologue of Epi-Vitamin D, in an embodiment is ¹³C₅-25-Hydroxy-Vitamin D3 epimer, ¹³C₅-Epi-Vitamin D, or ³H₅-Epi-vitamin D.

Embodiment 28. The method of any one of embodiments 1 to 27, wherein said analyte is Testosterone and, in an embodiment, the interferent is Epitestosterone.

Embodiment 29. The method of any one of embodiments 1 to 25 and 28, wherein said interferent monitoring compound is Epitestosterone-¹³C₃ or Epitestosterone-D₃,

Embodiment 30: The method of any one of embodiments 1 to 29, wherein said sample is a biological sample, in an embodiment is a sample of a subject, in a further embodiment a patient sample.

Embodiment 31. The method of any one of embodiments 1 to 30, wherein said method comprises further step d) providing a verified analyte measurement based on comparison step c).

Embodiment 32: The method of any one of embodiments 1 to 31, wherein a verified analyte measurement is a measurement for which the risk of interference by a non-analyte compound is assessed or can be assessed, in an embodiment quantified.

Embodiment 33: The method of any one of embodiments 1 to 32, wherein said chromatography is liquid chromatography, gas chromatography, capillary electrophoresis chromatography, and/or ion mobility chromatography, in an embodiment is liquid chromatography. Embodiment 34: The method of any one of embodiments 1 to 33, said method is a method of routine analyte measurement and/or of interference checking and/or interference monitoring.

Embodiment 35: The method of any one of embodiments 1 to 34, wherein in case said analyte measurement is not accepted, the analyte measurement value is not reported, the analyte measurement is flagged as rejected, and/or the chromatography device is flagged unsuitable for measurement of the analyte in the sample type.

Embodiment 36. A method of quality control of a chromatography mass spectrometry (MS) measurement of an analyte in a sample, comprising the steps
A) measuring the analyte in the sample using the chromatography mass spectrometer device and determining at least one chromatogram;
B) verifying the analyte measurement according to the method of any one of embodiments 1 to 35, and
C) evaluating quality of said chromatography-MS measurement based on the results of step B). Embodiment 37: The method of embodiment 36, wherein at least one of (i) flagging the result of step A) as unreliable; (ii) not outputting the result of step A), and (iii) flagging the chromatography device as unsuitable for measurement of the analyte in the sample type, is performed in case the analyte peak is rejected in step B).

Embodiment 38: The method according to any one of the preceding embodiments, wherein method steps B) and/or C), in an embodiment steps B) and C), are performed by a computer.

Embodiment 39: The method according to any one of the preceding embodiments, wherein said chromatography mass spectrometer device comprises a tandem mass spectrometer (MS/MS) unit.

Embodiment 40. A system for determining an amount of at least one analyte in a sample comprising:
(I) at least one chromatography mass spectrometer device, wherein the chromatography mass spectrometer device is configured for measuring the analyte in the sample and for acquiring data points over time, in an embodiment for performing step b) of a method according to any one of embodiments 1 to 39; and
(II) at least one evaluation device, wherein the evaluation device is configured for performing at least step c) of the method according to any one of embodiments 1 to 39.

Embodiment 41: The system of embodiment 40, wherein the system is configured to perform the method for quality control according to any one of embodiments 36 to 39.

Embodiment 42: A computer or computer network comprising at least one processor, wherein the processor is adapted for performing at least steps b), c) and/or d), in an embodiment all steps b) to d) of the method according to any one of embodiments 1 to 39.

Embodiment 43: A computer loadable data structure that is adapted to perform at least steps c) and/or d), in an embodiment all steps c) to d) of the method according to any one of embodiments 1 to 39 while the data structure is being executed on a computer.

Embodiment 44: A computer program, wherein the computer program is adapted to perform at least steps c) and/or d), in an embodiment all steps c) to d) of the method according to any one of embodiments 1 to 39 while the data structure is being executed on a computer.

Embodiment 45: A computer program comprising program means for performing at least steps c) and/or d), in an embodiment all steps c) to d) of the method according to any one of embodiments 1 to 39 while the data structure is being executed on a computer or on a computer network

Embodiment 46: A computer program comprising program means according to the preceding embodiment, wherein the program means are stored on a storage medium readable to a computer.

Embodiment 47: A storage medium, wherein a data structure is stored on the storage medium and wherein the data structure is adapted to perform at least steps c) and/or d), in an embodiment all steps c) to d) of the method according to any one of embodiments 1 to 39 after having been loaded into a main and/or working storage of a computer or of a computer network.

Embodiment 48: A computer program product having program code means, wherein the program code means can be stored or are stored on a storage medium, for performing at least steps steps c) and/or d), in an embodiment all steps c) to d) of the method according to any one of embodiments 1 to 39 when the program code means are executed on a computer or on a computer network.

All references cited in this specification are herewith incorporated by reference with respect to their entire disclosure content and the disclosure content specifically mentioned in this specification.

### FIGURE LEGENDS

FIG. 1: Schematic representation of a result of Example 1: addition of internal standard (Testosterone-D3) and interferent monitoring compound (Epitestosterone-¹³C3), measuring three different transitions; x-axis: time; y-axes: relative intensity of the respective transition.

FIG. 2: Schematic representation of a result of Example 2: addition of intemal standard (Testosterone-D3) and interferent monitoring compound (Epitestosterone-D3), measuring of two different transitions; x-axis: time; y-axes: relative intensity of the respective transition.

FIG. 3: Schematic representation of a result of Example 3: addition of A) Epitestosterone-D3, B) Epitestosterone-¹³C3 as interferent monitoring compound, measurement of two different transitions; x-axis: time; y-axes: relative intensity of the respective transition;.

FIG. 4: Schematic representation of a result of Example 4: addition of internal standard (Testosterone-D3), measurement of two different transitions; x-axis: time; y-axes: relative intensity of the respective transition.

FIG. 5: Schematic representation of a result of Example 5: addition of interferent monitoring compound (Epitestosterone): measurement of one transition; x-axis: time; y-axes: relative intensity of the respective transition.

### EXAMPLES

The following Examples shall merely illustrate the invention. They shall not be construed, whatsoever, to limit the scope of the invention.

### Example 1

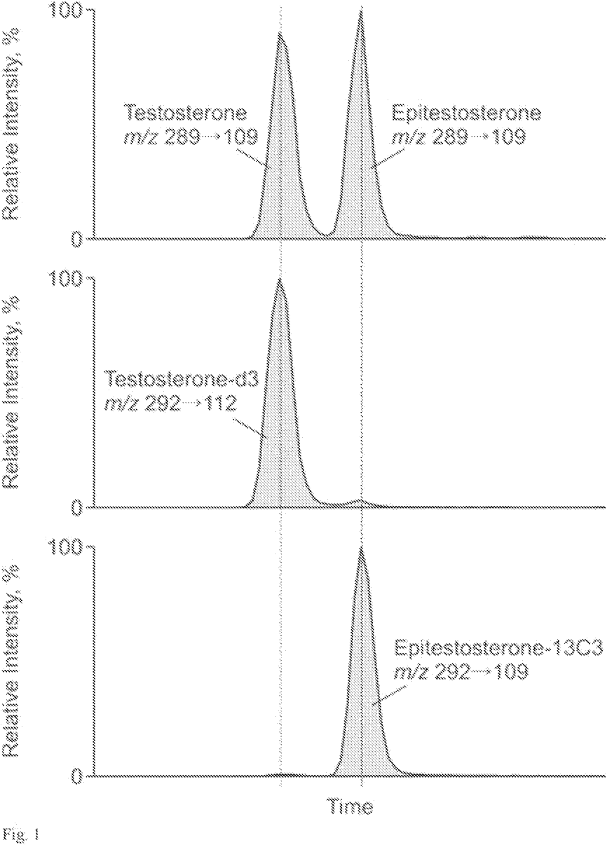

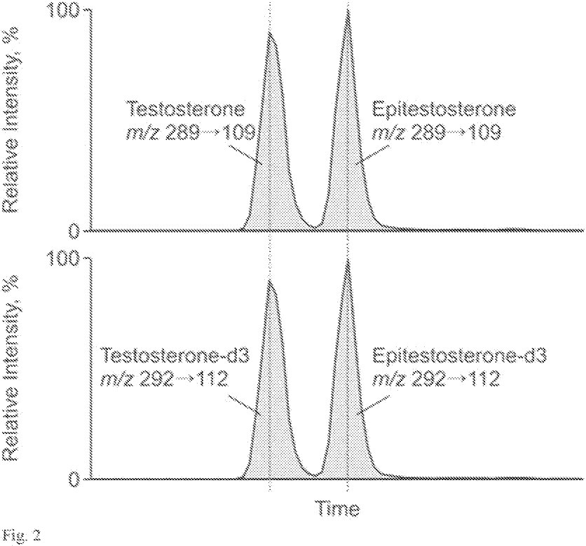

To a routine sample for LC-MS/MS measurement of testosterone, an internal standard (Testosterone-D3) and an interferent monitoring compound (Epitestosterone-¹³C3) were admixed. Intensities of m/z 289 -> 109, m/z 292 -> 112, and m/z 292 -> 109 transitions over LC progress were recorded; a possible result is schematically shown in FIG. 1.

By comparing properties of the m/z 292 -> 112 and m/z 292 -> 109 peaks, quality of separation of the internal standard from the interferent monitoring compound, which is a measure of separation between analyte and an interferent which may possibly be present, can be provided.

From the analyte peak of the m/z 289 -> 109 transition, optionally in combination with peak. of the internal standard m/z 292 -> 112 transition, the amount of testosterone can be derived.

### Example 2

To a routine sample for LC-MS/MS measurement of testosterone, an internal standard (Testosterone-D3) and an interferent monitoring compound (Epitestosterone-D3) were admixed. Intensities of m/z 289 -> 109 and m/z 292 -> 112 transitions over LC progress were recorded; a possible result is schematically shown in FIG. 2.

By comparing properties of the m/z 292 -> 112 peaks of internal standard and interferent monitoring compound, quality of separation of the internal standard from the interferent monitoring compound, which is a measure of separation between analyte and an interferent which may possibly be present, can be provided.

From the analyte peak of the m/z 289 -> 109 transition, optionally in combination with peak of the internal standard m/z 292 -> 112 transition, the amount of testosterone can be derived.

### Example 3

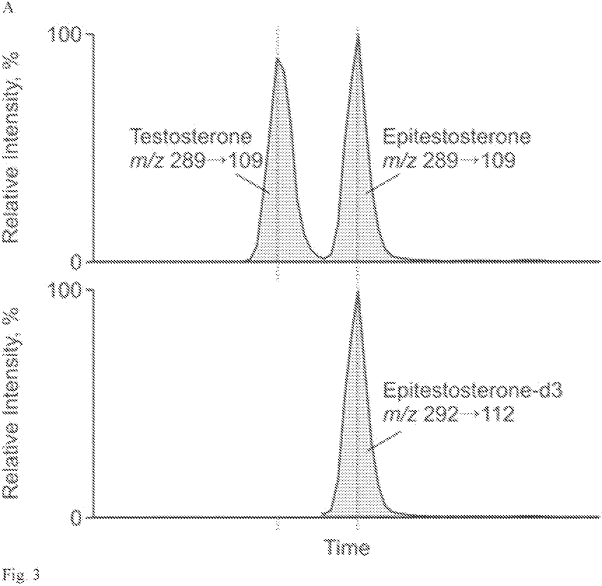

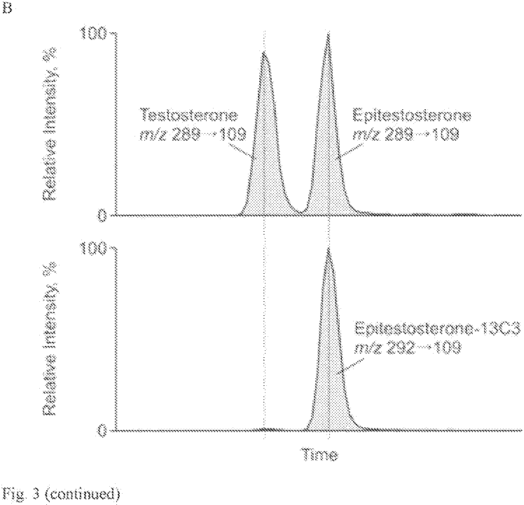

To a routine sample for LC-MS/MS measurement of testosterone, an internal standard (Testosterone-D3) and an interferent monitoring compound (Epitestosterone-D3 or Epitestosterone-¹³C3) was admixed. Intensities of m/z 289 -> 109 and (i) m/z 292 -> 112 transitions if Epitestosterone-D3 was used, or (ii) m/z 292 -> 109 transitions if Epitestosterone-¹³C3 was used, over LC progress were recorded; a possible result is schematically shown in FIGS. 3 A) and B).

By comparing properties of (i) the m/z 292 -> 112 peak or (ii) the m/z 292 -> 109 peak of the interferent monitoring compound to the analyte m/z 289 -> 109 peak, quality of separation of the analyte from the interferent monitoring compound, which is a measure of separation between analyte and an interferent which may possibly be present, can be provided.

From the analyte peak of the m/z 289 -> 109 transition, the amount of testosterone can be derived.

### Example 4

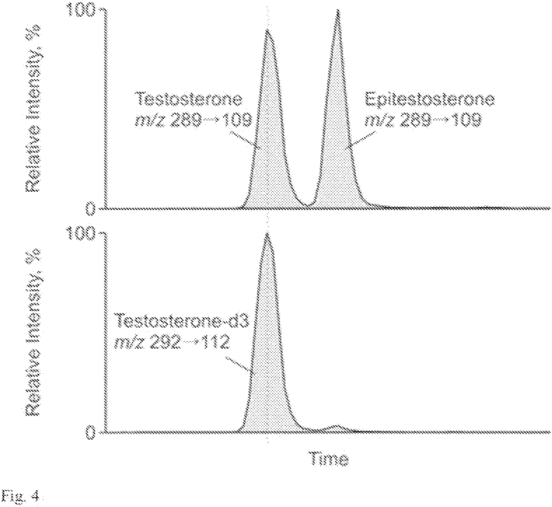

To a routine sample for LC-MS/MS measurement of testosterone, an internal standard (Testosterone-D3) and an interferent monitoring compound (Epitestosterone) were admixed. Intensities of m/z 289 -> 109 and m/z 292 -> 112 transitions over LC progress were recorded; a possible result is schematically shown in FIG. 4.

By comparing properties of the internal standard m/z 292 -> 112 peak to the interferent monitoring compound m/z 289 -> 109 peak, quality of separation of the internal standard from the interferent monitoring compound, which is a measure of separation between analyte and an interferent which may possibly be present, can be provided.

From the analyte peak of the m/z 289 -> 109 transition, optionally in combination with the the internal standard m/z 292 -> 112 transition peak, the amount of testosterone can be derived.

### Example 5

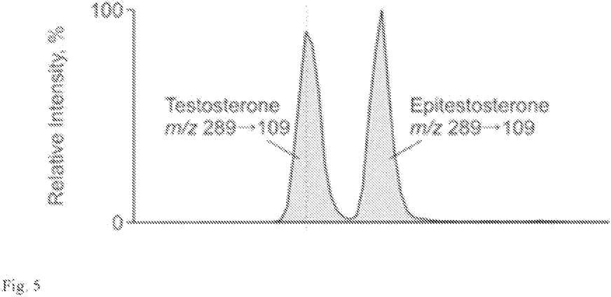

To a routine sample for LC-MS/MS measurement of testosterone, an interferent monitoring compound (Epitestosterone) was admixed. Intensities of the m/z 289 -> 109 transition over LC progress was recorded; a possible result is schematically shown in FIG. 5.

By comparing properties of the analyte m/z 292 -> 112 peak to the interferent monitoring compound m/z 292 -> 112 peak, quality of separation of the analyte from the interferent monitoring compound, which is a measure of separation between analyte and an interferent which may possibly be present, can be provided.

From the analyte peak of the m/z 289 -> 109 transition the amount of testosterone can be derived.

1. A method for providing a verified analyte measurement of a sample with a chromatography mass spectrometer device, said method comprising :
a) admixing an interferent monitoring compound to the sample;
b) determining a chromatogram of the sample by acquiring a plurality of data points for signal intensities over time for said interferent monitoring compound, said analyte ; and
c) comparing a property of an interferent monitoring compound peak to a property of an analyte peak.

2. The method of claim 1, wherein said interferent monitoring compound is an isotopologue of an interferent.

3. The method of claim 1, wherein in step b) the chromatogram is determined based on signals being (I) non-identical between the analyte and the interferent monitoring compound; or (II) identical between the analyte and the interferent monitoring compound.

4. The method of claim 1, wherein
A) in step a) an interferent monitoring compound and an internal standard are admixed to the sample; and wherein said interferent monitoring compound and said internal standard are isotope-labelled, said;
B) in step b) the chromatogram is determined based on signals being non-identical between the analyte, the internal standard, and the interferent monitoring compound; and
C) wherein in step c) the property of the internal standard peak is compared to the property of the interferent monitoring compound peak.

5. The method of claim 1, wherein
A) in step a) an interferent monitoring compound and an internal standard are admixed to the sample; wherein said interferent monitoring compound and said internal standard are isotope-labelled said;
B) in step b) the chromatogram is determined based on signals being identical between the internal standard and the interferent monitoring compound and non-identical between the analyte and the internal standard; and
C) wherein in step c) the property of the internal standard peak is compared to the property of the interferent monitoring compound peak.

6. The method of claim 1, wherein
A) in step a) an interferent monitoring compound is admixed to the sample; wherein said interferent monitoring compound is isotope-labelled, said compound;
B) in step b) the chromatogram is determined based on signals being non-identical between the analyte and the interferent monitoring compound; and
C) wherein in step c) the property of the analyte peak is compared to the property of the interferent monitoring compound peak; or wherein
A) in step a) an interferent monitoring compound and an internal standard are admixed to the sample; wherein said internal standard is isotope-labelled said;
B) in step b) the chromatogram is determined based on signals being non-identical between the internal standard and the interferent monitoring compound; and
C) wherein in step c) the property of the internal standard peak is compared to the property of the interferent monitoring compound peak; or wherein
A) in step a) an interferent monitoring compound is admixed to the sample;
B) in step b) the chromatogram is determined based on signals being identical between the analyte and the interferent monitoring compound; and
C) wherein in step c) the property of the analyte peak is compared to the property of the interferent monitoring compound peak.

7. The method of claim 1, wherein said method further comprises performing peak identification of at least one interferent monitoring compound peak and at least one analyte peak.

8. The method of claim 1, wherein said comparing in step c) comprises determining a resolution between the analyte peak and the interferent monitoring compound peak, wherein, resolution is calculated based on the retention times and the full width at half maximum values of the respective peaks according to:
$$R = 2 \frac{{t}_{2} - {t}_{1}}{{w}_{2} + {w}_{1}}$$
with R = resolution,
t₁ = retention time of the first peak,
t₂ = retention time of the second peak,
w₁ = full width at half maximum of the first peak; and
w₂ = full width at half maximum of the second peak.

9. The method of claim 1, wherein said method comprises additional step d) providing a verified analyte measurement based on comparison step c), wherein, the analyte measurement is accepted in case the resolution between the analyte peak and the interferent monitoring compound peak or between the internal standard peak and the interferent monitoring compound peak is higher than 1.5 .

10. The method of claim 1, wherein said interferent is an isomer of the analyte .

11. The method of claim 1, wherein said analyte is Vitamin D and the interferent is Epi-Vitamin D said.

12. The method of claim 1, wherein said method is a method of routine analyte measurement and/or of interference checking and/or interference monitoring.

13. A method of quality control of a chromatography mass spectrometry (MS) measurement of an analyte in a sample, comprising:
A) measuring the analyte in the sample using the chromatography mass spectrometer device and determining at least one chromatogram;
B) verifying the analyte measurement according to the method of claim 1, and
C) evaluating quality of said chromatography-MS measurement based on the results of step B).

14. A system for determining an amount of at least one analyte in a sample comprising:
(I) at least one chromatography mass spectrometer device, wherein the chromatography mass spectrometer device is configured for performing step b) of a method according to claim 1; and
(II) at least one evaluation device, wherein the evaluation device is configured for performing at least step c) of the method according to claim 1.

15. (canceled)

16. The method of claim 9, wherein the interferent monitoring compound peak is higher than 2.

17. The method of claim 10, wherein said interferent is a stereoisomer of the analyte.

18. The method of claim 10, wherein said interferent is a diastereomer, an enantiomer, or a cis-trans isomer.

19. The method of claim 1, wherein said analyte is Testosterone and the interferent is Epitestosterone.

20. The method of claim 1, wherein step a) comprises admixing an interferent monitoring compound and an internal standard to the sample;
step b) comprises determining a chromatogram of the sample by acquiring a plurality of data points for signal intensities over time for said interferent monitoring compound, said analyte, and said internal standard; and
step c) comprises comparing a property of an interferent monitoring compound peak to a property of an internal standard peak and/or to a property of an analyte peak.

21. The method of claim 20, wherein said internal standard is an isotopologue of the analyte.

22. The method of claim 20, wherein in step b) the chromatogram is determined based on signals being (I) non-identical between the analyte, the internal standard, and the interferent monitoring compound; (II) identical between the internal standard and the interferent monitoring compound and non-identical between the analyte and the internal standard, (III) non-identical between the analyte and the interferent monitoring compound, (IV) non-identical between the internal standard and the interferent; or (V) identical between the analyte and the interferent monitoring compound.
